(19) United States
(12) Patent Application Publication
(10) Pub. No.: US 2017/0159037 A1
(43) Pub. Date: Jun. 8, 2017

(54) COMMERCIAL PRODUCTION OF ALLERGEN AMB A 1 BY MEANS OF TRANSIENT EXPRESSION IN PLANTS

(71) Applicant: ANGANY GENETICS, VAL DE REUIL (FR)

(72) Inventors: Veronique GOMORD, ROUEN (FR); Anne-Catherine FITCHETTE, PAVILLY (FR); Virginie CATALA, ROUEN (FR); Loic FAYE, ST JACQUES SUR DARNETAL (FR)

(73) Assignee: Angany Genetics, Val de Reuil (FR)

(21) Appl. No.: 15/313,802

(22) PCT Filed: May 20, 2015
(86) PCT No.: PCT/FR2015/051319

§ 371 Date: Nov. 23, 2016

(30) Foreign Application Priority Data
May 23, 2014 (FR) 14 54653

(51) Int. Cl. C12N 9/88

(52) U.S. Cl. CPC C12N 9/88; C12N 2510/02; C12N 2511/00; C12N 2800/22; C12N 2820/55

(57) ABSTRACT

A plant cell including a DNA molecule having at least one heterologous nucleotide sequence encoding a preproprotein of a pectate lyase chosen from Amb a 1, the alpha subunit of Amb a 1, the beta subunit of Amb a 1, and homologs of Amb a 1, functionally bound to a strong promoter.

The present invention relates to a plant cell comprising a DNA molecule comprising at least one heterologous nucleotide sequence encoding a preproprotein of a pectate lyase chosen from Amb a 1, the alpha subunit of Amb a 1, the beta subunit of Amb a 1, and homologs of Amb a 1, functionally bound to a strong promoter.

The invention also relates to a process for producing pectate lyase using the plant cell. Ragweed, in particular Ambrosia artemisiifolia, is an annual plant which belongs to the Compositae tubuliflorae (or Asteraceae) family This wild plant which is native to north America flowers at the end of summer until fall. Its pollen has very high allergenicity. Thus, ragweed is the primary cause of pollen allergy in the United States, with approximately 30 million people out of 40 million suffering from pollenoses. Ragweed was unintentionally introduced to France in the nineteenth century and is a highly invasive plant which has rapidly spread from the Rhône-Alpes region to the north of France. Thus, new ragweed growth was found in Ile de France in 2011.

In sensitive people, the pollen of this plant causes an allergic reaction characterized by its severity. Ragweed allergic rhinitis is overall much more severe than that caused by other pollens. The symptoms, such as pruritus, anosmia (loss or reduction in sense of smell), rhinorrhea, sneezing and nasal blockages, are much greater. Moreover, in 50% of cases, this allergy leads to asthma, which is often more severe than that caused by other pollens. These symptoms are all the more marked the higher the pollen count in the air.

In the Rhône-Alpes region, ragweed allergy currently affects 6 to 12% of the population between the end of August and the start of September. Thus, just in this region, the total number of consumers of ragweed allergy medications increased significantly (+60%) between 2008 and 2011, going from 161 200 to 258 700 people, and the health spending caused by this allergy represents between 5.6 and 8.6 million euros. Ragweed allergy therefore currently represents a real public health problem. The simplest way to protect oneself from the risks of allergy would be to avoid exposure to ragweed pollen, but total avoidance already seems impossible despite prefectural orders making it compulsory to destroy ragweed. These measures have not made it possible to stop the spread of this invasive plant into a large number of regions.

“Symptomatic” treatments, administrated either locally (drops, eyewash, sprays) or generally (tablets, capsules), make it possible to relieve symptoms during the period in which the pollen is present in the air. However, symptoms rapidly return once treatment is stopped. Unlike “symptomatic” treatments, allergy immunotherapy (or desensitization) is the only treatment able to modify the evolution of the allergy illness. This therapeutic route makes it possible to treat the cause of the allergy, unlike “symptomatic” medications. However, as for other allergies, the extracts currently used for desensitizing patients allergic to ragweed are of insufficient quality for this treatment to be effective. Several ragweed allergens have been characterized, in particular Amb a 1 which is responsible for more than 80% of the cases of ragweed allergy. Various attempts to produce recombinant Amb a 1 intended for specific desensitization have failed. This is because Amb a 1 is a complex plant protein having post-translational maturations typical of plants, and only a plant expression system will be capable of producing this allergen in a useable and effective form for allergy immunotherapy.

There is therefore a need to produce this allergen Amb a 1 in recombinant form, with good quality, reproducibly and efficiently (with a good yield). “Good quality” means that the allergen is very highly similar, in its sequence and its structure, to the allergen naturally present in ragweed. There is also a need to produce this allergen at commercially and economically acceptable levels.

Surprisingly, the inventors have now discovered that although the production of the Amb a 1 allergen is toxic in vivo, it is possible in recombinant form by transient expression in plants. This production, which is carried out in a plant cell, makes it possible to very simply and reproducibly obtain the allergen Amb a 1 in the mature and active form, with good yield. In addition, the allergen Amb a 1 obtained is not contaminated by proteases and/or other contaminants of plant origin. The inventors were therefore able to obtain production of the allergen Amb a 1 at commercially acceptable levels.

A subject of the invention is therefore a plant cell comprising a DNA molecule comprising at least one heterologous nucleotide sequence encoding a preproprotein of a pectate lyase chosen from Amb a 1, the alpha subunit of Amb a 1, the beta subunit of Amb a 1, and homologs of Amb a 1, functionally bound to a strong promoter, preferably a 35S promoter. The DNA molecule bound to the strong promoter enables transient expression of the mature and active pectate lyase in the cell. Said plant cell is preferably a cell of Nicotiana benthamiana. The plant cell according to the invention preferably comprises a DNA molecule comprising at least one heterologous nucleotide sequence encoding a preproprotein of pectate lyase, functionally bound to a strong promoter, preferably a 35S promoter, said DNA molecule not being integrated into the plant cell genome.

The pectate lyase chosen from Amb a 1, the alpha subunit of Amb a 1, the beta subunit of Amb a 1, and homologs of Amb a 1, is referred to as “pectate lyase according to the invention” in the present application.

Another subject of the invention is a plant comprising at least one plant cell according to the invention.

Another subject of the invention is to provide a process for producing a pectate lyase according to the invention, comprising the expression of said pectate lyase in a plant cell according to the invention, or in a plant comprising such a cell.

Another subject of the invention relates to a pectate lyase obtainable by the process according to the invention, with a plant cell according to the invention or with a plant according to the invention. The pectate lyase obtained in this way may be used as medication. It may also be used in allergy immunotherapy, alone or in combination with at least one other Asteraceae allergen. It may also be used in allergy diagnosis, and be integrated into a kit for allergy diagnosis.

The plant cell according to the invention comprises a DNA molecule functionally bound to a strong promoter, preferably a 35S promoter. “Functionally bound” means that the DNA molecule is fused with the strong promoter, such that the strong promoter induces the transcription of the DNA molecule.

This enables the transient expression of said DNA molecule, i.e. without integration of the DNA, especially the cDNA, into the genome of the plant cell. Indeed, the use of transient expression in a plant cell or a plant according to the invention makes it possible to increase the production yields of the pectate lyase according to the invention, in active form, to high levels compatible with commercial use but incompatible with the survival of a plant if it were to express these toxic pectate lyases stably. In the case of transient expression, the plant biomass is harvested during the peak expression of the recombinant protein, i.e. typically 4 to 6 days after transfection.

The plant cell according to the invention preferably comprises an expression vector comprising at least one heterologous nucleotide sequence encoding a preproprotein of a pectate lyase according to the invention, functionally bound to a strong promoter, preferably a 35S promoter.

Amb a 1 is a pectate lyase originating from Ambrosia artemisiifolia. This allergen is recognized by more than 80% of patients sensitized to ragweed and is responsible for more than 90% of the allergenic activity of ragweed pollen. It is a 38 kDa protein, described as non-glycosylated, belonging to the pectate lyase family Twelve isoforms of Amb a 1 have been listed, from Amb a 1.0101 to Amb a 1.0502 (according to the site www.allergome.org).

The nucleic acid and amino acid sequences of the twelve isoforms are detailed in table 1 below:

TABLE 1
the twelve isoforms of Amb a 1, their nucleic
acid sequences and protein sequences

| | Accession no. | Accession no. |
|---|---|---|
| | (GenBank) | (UniProt) |
| | Nucleic acid | Amino acid |
| Isoallergens | sequence | sequence |
| Amb a 1.0101 | M80558 | P27759 |
| Amb a 1.0201 | M62981 | P27760 |
| Amb a 1.0202 | FR669658 | E1XUL3 |
| Amb a 1.0301 | M62961 | P27761 |
| Amb a 1.0302 | | P27761 (variant L48Y) |
| Amb a 1.0303 | M80560 | P27761 (variant H392R) |
| Amb a 1.0304 | FR669659 | E1XUL4 |
| Amb a 1.0305 | FR669660 | E1XUL5 |
| Amb a 1.0401 | M80562 | P28744 |
| Amb a 1.0402 | FR669664 | E1XUL9 |
| Amb a 1.0501 | M80561 | P27762 |
| Amb a 1.0502 | FR669666 | E1XUM1 |

“Amb a 1” according to the invention means the twelve isoforms mentioned in the table 1 above. The protein sequences described in this table 1 correspond to the preproproteins of the different isoforms.

Amb a 1 undergoes proteolysis in the pollen grain and/or during the process of extraction or of purification, resulting in two chains: alpha (26 kDa) and beta (12 kDa), connected non-covalently (King et al., 1974, 1981). It has been demonstrated that chemical modifications to Amb a 1, including the reduction and alkylation of the disulfide bridges, and also the process of denaturing/renaturing by urea or succinylation of the lysine residues, reduces its reactivity with regard to IgEs (King, 1976; Smith et al., 1988). The alpha and beta sub-chains of Amb a 1 have different reactivity to IgEs and T lymphocytes. Indeed, Amb a 1 beta contains a large number of IgE-binding epitopes, while Amb a 1 alpha acts as a hypoallergen and stimulates T cell activity.

Preferably, the protein sequence of Amb a 1 is SEQ ID NO: 7 (preproprotein). Preferably, the protein sequence of the beta subunit is SEQ ID NO: 8. Preferably, the protein sequence of the alpha subunit is SEQ ID NO: 9.

“Homologs of Amb a 1” means proteins of the Asteraceae family which have at least 57% identity, preferably at least 60% identity, preferably at least 64% identity, with one of the isoforms of Amb a 1. The homolog of Amb a 1 is preferably a protein of a plant of the genus Ambrosia or Artemisia, more preferentially of Artemisia vulgaris, Ambrosia psilostachya or Ambrosia trifida, which has at least 57% identity, preferably at least 60% identity, preferably at least 64% identity, with one of the isoforms of Amb a 1. The homolog of Amb a 1 is preferably chosen from Amb p 1 (from Ambrosia psilostachya, accession code 9064 on www.allergome.org) and Art v 6 (from Artemisia vulgaris, accession number AOPJ16 in Uniprot).

Within the meaning of the present invention, “percentage identity” between two amino acid sequences is intended to denote a percentage of identical amino acid residues between the two sequences to be compared, obtained after the best alignment, this percentage being purely statistical and the differences between the two sequences being distributed randomly over their whole length. “Best alignment” or “optimal alignment” means the alignment for which the percentage identity determined as below is the highest. Sequence comparisons between two amino acid sequences are traditionally carried out by comparing these sequences after having aligned them optimally, said comparison being carried out by segment or “comparison window” to identify and compare local regions of sequence similarity. The optimal alignment of the sequences for the comparison may be carried out, aside from manually, by means of the local homology algorithm of Smith and Waterman (1981, J. Mol Evol., 18:38-46), by means of the local homology algorithm of Needleman and Wunsch (1970), by means of the similarity searching method of Pearson and Lipman (1988, PNAS, 85: 2444-2448), or by means of computer software using these algorithms (GAP, BESTFIT, BLAST P, BLAST N, FASTA and TFASTA in the Wisconsin Genetics Software Package, Genetics Computer Group, 575 Science Dr., Madison, Wis.).

Thus, the pectate lyase according to the invention is preferably chosen from Amb a 1.0101, Amb a 1.0201, Amb a 1.0202, Amb a 1.0301, Amb a 1.0302, Amb a 1.0303, Amb a 1.0304, Amb a 1.0305, Amb a 1.0401, Amb a 1.0402, Amb a 1.0501, Amb a 1.0502, the alpha subunit of Amb a 1, the beta subunit of Amb a 1 and proteins of the Asteraceae family which have at least 57% identity, preferably at least 60% identity, preferably at least 64% identity, with one of the isoforms of Amb a 1.

More preferentially, the pectate lyase according to the invention is chosen from Amb a 1.0101, Amb a 1.0201, Amb a 1.0202, Amb a 1.0301, Amb a 1.0302, Amb a 1.0303, Amb a 1.0304, Amb a 1.0305, Amb a 1.0401, Amb a 1.0402, Amb a 1.0501, Amb a 1.0502, the alpha subunit of Amb a 1, the beta subunit of Amb a 1 and proteins of Artemisia vulgaris, Ambrosia psilostachya or Ambrosia trifida which have at least 57% identity, preferably at least 60% identity, preferably at least 64% identity, with one of the isoforms of Amb a 1.

The DNA molecule comprises at least one heterologous nucleotide sequence encoding a preproprotein of the pectate lyase according to the invention.

The preproprotein of the pectate lyase according to the invention comprises a signal peptide, a propeptide and the mature pectate lyase. The heterologous nucleotide sequence encoding the preproprotein according to the invention therefore comprises a sequence encoding the signal peptide, a sequence encoding the propeptide and a sequence encoding the mature pectate lyase. The heterologous nucleotide sequence encoding the preproprotein according to the invention preferably comprises a sequence encoding the signal peptide, a sequence encoding the propeptide of the beta subunit, a sequence encoding the beta subunit, a sequence encoding the propeptide of the alpha subunit and a sequence encoding the alpha subunit.

The signal peptide is especially any signal peptide recognized by the plant cell, whether originating from a pectate lyase or not. It especially targets the pectate lyase according to the invention in the intercellular medium. The signal peptide is preferably that of tobacco chitinase or the natural pectate lyase signal peptide.

The propeptide is for its part the natural propeptide of a pectate lyase or else the propeptide of a peptidase C1A. In a preferred embodiment, the propeptide is a propeptide of peptidases C1A, especially from mites or of plant origin. The propeptide of peptidase C1A is preferably the natural propeptide or the mutated or unmutated propeptide of Der p 1. The propeptide of peptidase C1A is preferably chosen from the natural propeptide of peptidase C1A and the sequence SEQ ID NO: 6 (i.e. the amino acids 19 to 98 of the Uniprot sequence p08176). In a preferred embodiment of the present invention, the pectate lyase according to the invention is produced in fusion with its own propeptide or in fusion with the propeptide of mite peptidase C1A or in fusion with a propeptide of plant peptidase C1A in order to increase the protein production yields.

The sequence of the preproprotein of pectate lyase is preferably chosen from the protein sequences listed in table 1 above and the sequences having at least 57%, preferably at least 60%, preferably at least 64% identity with one of the latter.

The heterologous nucleotide sequence encoding the preproprotein is preferably chosen from SEQ ID NOs: 1 to 5. Its sequence is preferably SEQ ID NO: 1, SEQ ID NO: 2, SEQ ID NO: 3, SEQ ID NO: 4 or SEQ ID NO: 5.

According to the invention, the heterologous nucleotide sequence encoding the preproprotein may be obtained from an already cloned known gene encoding a pectate lyase according to the invention, or else by screening a library of cDNA with anti-pectate lyase antibodies. The methods used are well known to those skilled in the art and include especially identification of the gene by hybridization with probes, PCR, sequencing and molecular cloning. It is also possible to synthesize the gene to reflect the use of the codons preferred in plants; in this case, reference is made to codon optimization, which is often useful for strong expression of the selected proteases (Murray et al., Nucleic Acid Res. 17:477 498 (1980)).

In a preferred embodiment of the invention, the heterologous nucleotide sequence encoding the preproprotein also comprises a sequence referred to as intracellular trafficking sequence. This sequence makes it possible to target a compartment for the storage of the pectate lyase according to the invention, in order to control the maturation thereof. This maturation is necessary for obtaining pectate lyases in the most suitable form for allergy diagnosis and immunotherapy. Preferably this trafficking peptide sequence targets the peptide lyase according to the invention in soluble or membrane form to the endoplasmic reticulum or the various compartments which constitute the endomembrane secretory system of the plant cell.

Once the gene of interest has been isolated and modified to contain all or part of the modifications described above and obtain the heterologous nucleotide sequence, the latter is placed in an expression vector by conventional methods. The selection of a suitable expression vector will depend on the method of introduction of the expression vector into the host cells. A typical expression vector contains elements of prokaryotic DNA encoding a bacterial origin of replication and a gene for antibiotic resistance to provide for the growth and selection of the expression vector in the bacterial host, a cloning site for the insertion of an exogenous DNA sequence encoding the pectate lyase, eukaryotic DNA elements such as a sequence for initiating transcription of the exogenous gene, such as a promoter, and DNA elements which control transcript processing, such as termination/polyadenylation sequences, and an expression cassette allowing the expression of a silencing suppressor. It also contains sequences such as tDNAs which are necessary for the integration of a length of DNA into the plant or into the plant cell.

The expression vector preferably comprises:
elements of prokaryotic DNA encoding a bacterial origin of replication and a gene for antibiotic resistance;
at least one heterologous nucleotide sequence encoding a preproprotein of a pectate lyase according to the invention, functionally bound to a strong promoter, preferably a 35S promoter;
an expression cassette allowing the expression of a silencing suppressor, preferably p19; and
DNA elements which control transcript processing, such as termination/polyadenylation sequences, preferably the Tnos sequence (nopaline synthase termination sequence).

The expression vector is preferably pAG01.

The promoters used to control the expression of the pectate lyase are strong promoters, and may be promoters of plant genes, such as, for example, the ubiquitin promoter, the ribulose-1,5-bisphosphate carboxylase small subunit promoter, Agrobacterium tumefaciens promoters, nopaline synthase and octopine synthase promoters, or else viral promoters such as the 19S and 35S of cauliflower mosaic virus (CaMV). The strong promoter is preferably 35S.

The high expression and the quality of the recombinant pectate lyases produced in the invention make it possible to devise their commercial production. Indeed, the pectate lyase according to the invention is typically expressed in an amount equal to at least 0.1% of the total soluble proteins of the plant, but often in a much higher amount which may reach 5 to 10%.

The plant comprising the plant cells according to the invention may be a whole plant but may also be part of a plant such as leaves.

The plant or the plant cells according to the invention may be used as such, as medication.

The plant or the plant cells according to the invention may be used as such, for applications such as biofuel, in animal feed, or else in the production of paper or textile (cotton fibres especially).

In other applications, the recombinant pectate lyase is purified after extraction from the plant or the plant cells expressing it. In order to facilitate its purification, the enzyme may be expressed in fusion with tags (His6, GST, MBP, FLAG etc.) which will preferably be located at the N-terminal or C-terminal position of the mature protein.

The pectate lyase according to the invention, obtainable by the process according to the invention, with a plant cell according to the invention or with a plant according to the invention, may be used as medication. It may also be used in allergy immunotherapy, alone or in combination with at least one other Asteraceae allergen. This other Asteraceae allergen may be chosen from Amb a 11, Amb a 5 and mixtures thereof.

It may also be used in the diagnosis of allergies, especially pollen allergies. Another subject of the invention is therefore a kit for the diagnosis of allergies, comprising the pectate lyase according to the invention, and means for measuring antibodies directed against this protein. Another subject of the invention is a kit for the diagnosis of allergies, comprising the pectate lyase according to the invention, means for measuring antibodies directed against this protein, and means for measuring antibodies directed against another Asteraceae allergen, for example the allergen Amb a 11 or Amb a 5.

The general methods for culturing plants, and also the processes for introducing expression vectors into a plant tissue, are available to those skilled in the art. They are varied and depend on the plant selected. The plants will preferably be cultivated according to the techniques specific to the Allergopur platform. This process for producing recombinant proteins is described in application FR1255510 and comprises a first step of culturing the plant cell or plant aeroponically or hydroponically, preferably culturing on free floats and under LED lighting. After this first step, especially five weeks of hydroponic culture on free floats, the plants are agroinfiltrated under vacuum by agrobacteria comprising a DNA fragment encoding the pectate lyase according to the invention. This step of agroinfiltration may be carried out by any means enabling a vacuum to be created. In the process used according to the invention it is preferably carried out under vacuum by the Venturi effect. Among the agrobacteria which may be used according to the invention, mention may preferably be made of the strains LBA4404, GV3101, EHA 101/105 or C58. According to a first alternative, once the agroinfiltration step has been carried out, the plants are typically drained upside-down for 15 minutes, then placed back in culture, typically for 3 to 6 days, ideally ensuring frequent atomization thereof for the first 6 hours of culture following the agroinfiltration. The protein is finally extracted and purified. The protein is preferably extracted and purified as described in application FR1255510. According to a second alternative, once the agroinfiltration step has been carried out, the plants are directly placed back in culture, typically for 3 to 6 days, then the protein is extracted and purified.

The protein may be extracted by grinding the leaves, or else according to a process which relies on enzymatic infiltration.

In such a process which relies on enzymatic infiltration, the protein is extracted by the following steps:
infiltration under vacuum (especially as was described above for agroinfiltration) of the aerial part of the plants, in an enzymatic solution comprising a pectinase or maceroenzyme, which does not have proteolytic activity. The pectinase is preferably P162L sold by Biocatalyst, formulated at 4% in a medium comprising 50mM sodium citrate pH 5.2, 0.5 M NaCl and 0.04% metabisulfite. The maceroenzyme is preferably formulated at 0.5% in a medium comprising 50mM sodium citrate pH 5.2, 0.5M NaCl and 0.04% metabisulfite,
taking off the infiltrated plant leaves and incubation in an enzymatic solution of pectinase or maceroenzyme, for a duration of between 2 h 30 and 5 h, preferably 3 h or 4 h 30, at a temperature of between 24° C. and 30° C., preferably 26° C., then
placing the mixture obtained under agitation at between 20 and 30 rpm at room temperature (i.e. approximately 20-23° C.) for a duration of between 30 minutes and 2 h,
the digestate is then filtered, preferably on a 250 um cloth, then optionally centrifuged (for example at 250 x g for 10 minutes),
the supernatant is recovered in order to carry out deep filtration, preferably on a K100 filter (sold by Pall). The filtrate is preferably concentrated 20 times on a 5 kDa cassette and its pH is adjusted to 7 with Na₃PO₄.

The allergens Amb a 1 and Amb a 11 may be purified by chromatography from extracts as was described in the application FR 1255510.

Thus, preferably, another subject of the invention is a process for producing a pectate lyase according to the invention in a plant cell or a plant, comprising the following steps:
a) transformation of agrobacteria with an expression vector comprising a heterologous nucleotide sequence encoding a preproprotein of a pectate lyase according to the invention, functionally bound to a strong promoter; and
b) transfection of the plant cell or of the plant with the agrobacteria obtained in step a).

Preferably, the agrobacteria which may be used in step a) are chosen from the strains LBA4404, GV3101, EHA 101/105 and C58. The expression vector used in step a) preferably comprises:
elements of prokaryotic DNA encoding a bacterial origin of replication and a gene for antibiotic resistance;
at least one heterologous nucleotide sequence encoding a preproprotein of a pectate lyase according to the invention, functionally bound to a strong promoter, preferably a 35S promoter;
an expression cassette allowing the expression of a silencing suppressor, preferably p19; and
DNA elements which control transcript processing, such as termination/polyadenylation sequences, preferably the Tnos sequence.

The transformation of step a) is typically carried out by methods known from the prior art, for example by thermal shocks with successive passes at 4° C., −80° C. and 37° C.

The transfection of step b) preferably comprises the following steps:

b1) culturing the plant cell or plant aeroponically or hydroponically, under LED lighting, preferably for five weeks hydroponically on free floats,

b2) agroinfiltration of the plant cell or plant obtained in b1), under vacuum, by the agrobacteria obtained in step a). This step of agroinfiltration is preferably carried out under vacuum by the Venturi effect.

b3) returning the plant cell or plant obtained in b2) to culturing, typically for 3 to 6 days.

Finally, the pectate lyase according to the invention produced in this way is preferably extracted and purified.

The process for producing a pectate lyase according to the invention in a plant cell or a plant preferably also comprises a step c) of extraction of the pectate lyase produced, said step c) comprising the following steps:
infiltration under vacuum of the plant cell or leaves (i.e. the aerial part) of the plant, in an enzymatic solution comprising a pectinase or maceroenzyme, which does not have proteolytic activity. The pectinase or maceroenzyme are preferably as described above,
taking off the infiltrated plant cell or infiltrated leaves from the plant and incubation, in an enzymatic solution of pectinase or maceroenzyme, for a duration of between 2 h 30 and 5 h, at a temperature of between 24° C. and 30° C., then
placing the mixture obtained under agitation at between 20 and 30 rpm at room temperature for a duration of between 30 minutes and 2 h,
filtration then optional centrifugation of the digestate obtained, and
recovery of the supernatant.

The following examples illustrate the invention without aiming to limit the scope thereof. It will be obvious to those skilled in the art that variants and modifications are possible and fall within the context and spirit of the invention.

The legends for the figures are as follows:

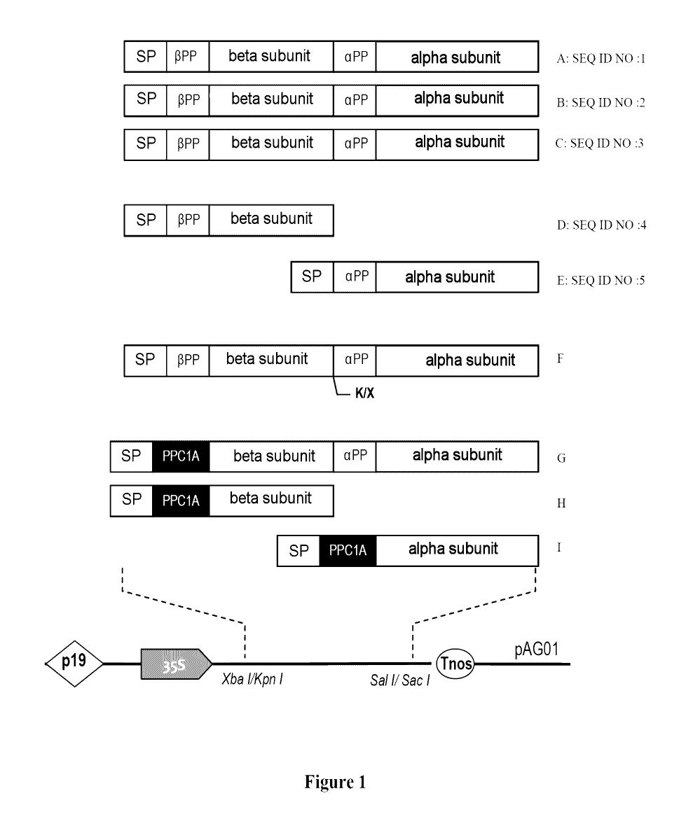

FIG. 1: Schematic representation of the different cassettes making it possible to produce a mature and active pectate lyase Amb a 1 (A, B, C, F and G) or the alpha subunit (E and I) or the beta subunit (D and H), using either the signal peptide and the natural propeptide of the protein (A-F) or the tobacco chitinase signal peptide and a propeptide of the peptidase CIA family (G-I) in order to increase yields.

The pectate lyase is also produced in a form mutated on lysine 180 (K180) in order to limit proteolysis enabling the production of the alpha and beta subunits (F).
A (SEQ ID NO: 1): cDNA encoding the natural preproprotein (of sequence SEQ ID NO: 7). This cDNA may be fused to trafficking signals described in application WO2008/056265,
B (SEQ ID NO: 2): cDNA optimized for use in N. benthamiana encoding the natural preproprotein (of sequence SEQ ID NO: 7). This cDNA may be fused to trafficking signals described in application WO2008/056265,
C (SEQ ID NO: 3): Harmonized cDNA encoding the natural form of the preproprotein (of sequence SEQ ID NO: 7). This cDNA contains codons optimized for use in N. benthamiana, but also comprises rare codons, in order to conserve the rhythm of synthesis of the protein for better preservation of the 3D structure. This cDNA may be fused to trafficking signals described in application WO2008/056265,
D (SEQ ID NO: 4): Native/optimized/harmonized cDNA encoding the beta subunit (of sequence SEQ ID NO: 8). This cDNA may be fused to trafficking signals described in application WO2008/056265.
E (SEQ ID NO: 5): Native/optimized/harmonized cDNA encoding the alpha subunit (of sequence SEQ ID NO: 9). This cDNA may be fused to trafficking signals described in application WO2008/056265.
F: Native/optimized/harmonized cDNA encoding the mutated (K180) form of the protein. This cDNA may be fused to trafficking signals described in application WO2008/056265.
G: Native/optimized/harmonized cDNA encoding the mature form of the protein fused to the tobacco chitinase signal sequence (Neuhaus, J.-M., 1996) and to the propeptide Der p1 (p08176-aa 19 to 98 or SEQ ID NO: 6). This cDNA may be fused to trafficking signals described in application WO2008/056265.
H: Native/optimized/harmonized cDNA encoding the beta subunit of the protein fused to the tobacco chitinase signal sequence (Neuhaus, J.-M., 1996) and to the propeptide Der p1 (p08176-aa 19 to 98 or SEQ ID NO: 6). This cDNA may be fused to trafficking signals described in application WO2008/056265.
I: Native/optimized/harmonized cDNA encoding the alpha subunit of the protein fused to the tobacco chitinase signal sequence (Neuhaus, J.-M., 1996) and to the propeptide Der p1 (p08176-aa 19 to 98 or SEQ ID NO: 6). This cDNA may be fused to trafficking signals described in application WO2008/056265.

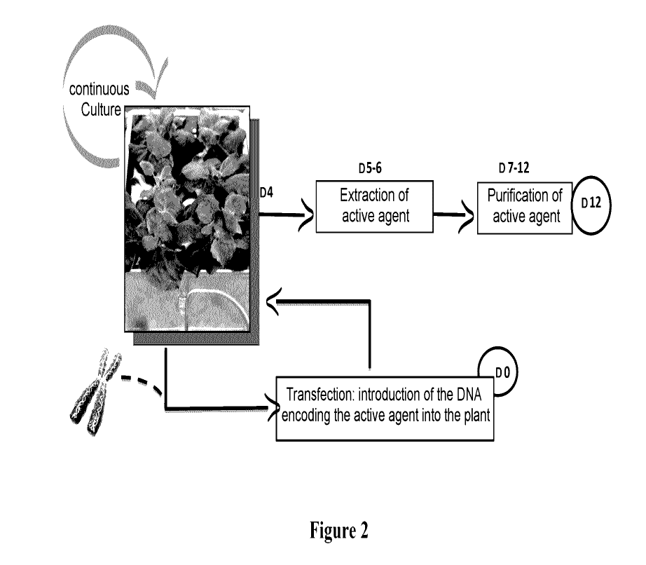

FIG. 2: The AllergoPur platform used for the expression and the production of the different forms of pectate lyase according to the invention.

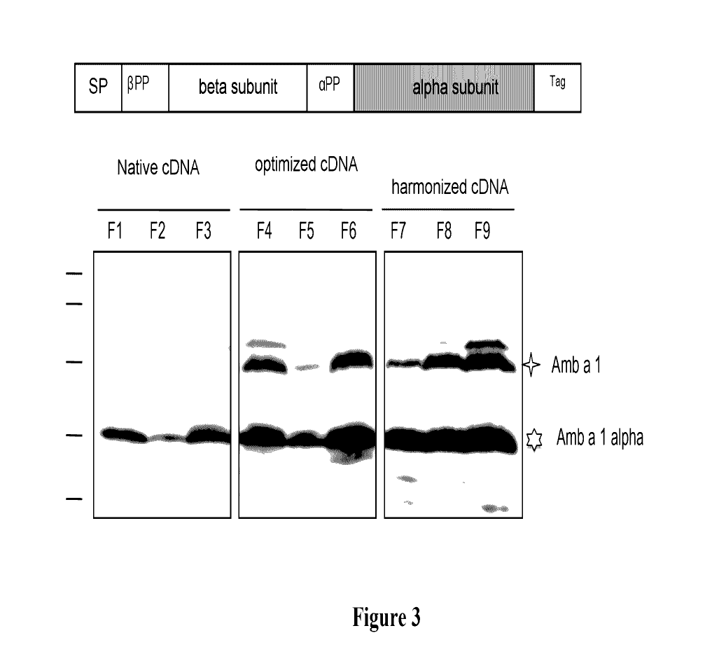

FIG. 3: Expression of the protein Amb a 1. The proteins extracted from plants transfected with the native cDNA (F1-F3), with the optimized cDNA (F4-F6) or with the harmonized cDNA (F7-F9) encoding the protein Amb a 1 were separated by SDS-PAGE and analyzed by immunodetection with an antibody directed against a Flag tag. The immunodetection analysis demonstrates the specific production of the protein Amb a 1 and of the alpha subunit. The cleaved beta subunit is not immunodetected on this blot.

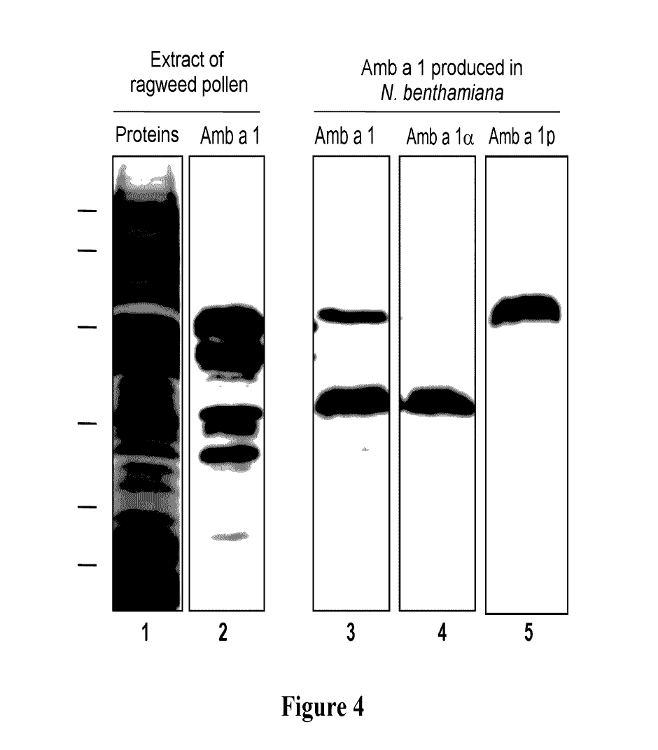

FIG. 4: Purification of the allergen Amb a 1. The proteins extracted from plants were transfected under vacuum with the harmonized cDNA encoding the protein Amb a 1 by IMAC chromatography. The fractions retained on the column were then analyzed by SDS-PAGE electrophoresis. Given the C-terminal position of the Flag, two polypeptides corresponding to the precursor form (preproprotein) (Amb a 1p) and to the alpha subunit are purified from the extract transfected with the total cDNA (lane 3). These two polypeptides (Amb a 1 alpha and Amb a 1p) are then separated (lanes 4 and 5, respectively) by molecular sieve chromatography. The two polypeptides produced have slightly lower electrophoretic mobility than the native proteins. This difference is explained by the presence of the flag at the C-terminal position.

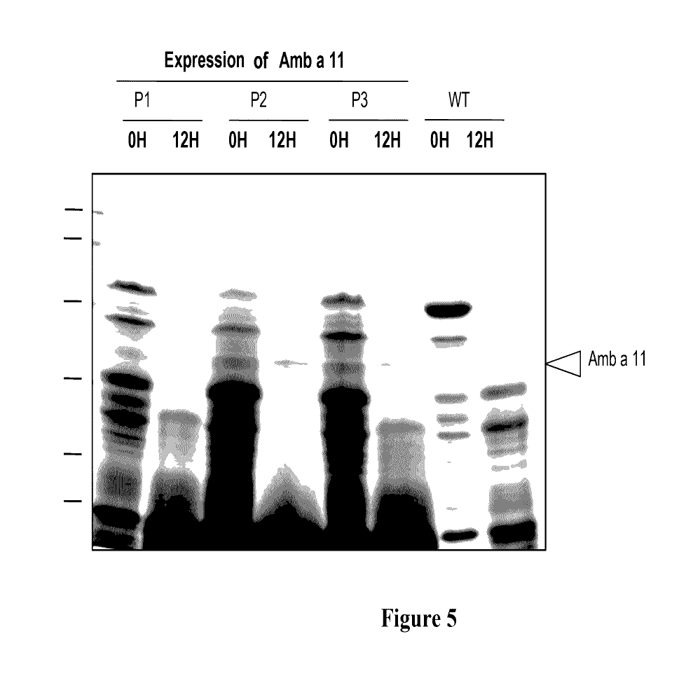

FIG. 5: Expression of the allergen Amb a 11. The proteins extracted from 3 plants (P1-P3) transfected with the cDNA encoding the allergen Amb a 11 were separated by SDS-PAGE electrophoresis. The protein extracts are analyzed as soon as they are extracted (0 H) or after 12 h incubation (12 H) at room temperature. The incubation at room temperature demonstrates the accumulation of two polypeptides corresponding to Amb a 11, and also the virtually total degradation of the N. benthamiana proteins. The allergen Amb a 11 is therefore produced in active form according to the same process as described presently for Amb a 1.

### EXAMPLE:

Molecular Design and Gene Synthesis

The cDNAs are synthesized in native form, optimizing the use of the codons for their recognition by the plant system or harmonizing the use of codons (reintroduction of rare codons to set the rhythm of the protein synthesis). Within the context of this invention, the preferred optimization is optimization for expression in Nicotiana benthamiana, as indicated in FIG. 1.

Preparation of Plasmids

Restriction sites Xba I/kpn I and Sal I/Sac I are respectively integrated at the 5′ and 3′ ends of the cDNA during synthesis. These sites are then used to clone the cDNAs in the binary expression vector pAG01 (FIG. 1). The cDNAs are cloned upstream of a 35S promoter (35S) and downstream of a nopaline synthase termination sequence (Tnos); the vector pAG01 also contains an expression vector enabling the expression of the silencing suppressor p19 simultaneously to the recombinant protein in order to increase the production yields. The vectors are then used to transform the strain LBA4404 of Agrobacterium tumefaciens.

Transient Expression of Pectate Lyases According to the Invention in Leaves of Nicotiana Benthamiana—Use of the AllergoPur Platform

For the production by transient expression, Agrobacterium tumefaciens LBA4404 is used for the transfer of a cDNA encoding pectate lyase without the gene of interest being integrated into the genome of the plant cell; reference is made here to transfection, not to transgenesis. The plants are cultivated in hydroponic conditions in the presence of a nutritive medium (GHE, floragrow, floramicro, florabloom, 10 ml/15 ml/5 ml per 10 l of osmosed water) and under LED lighting.

The agrobacterium is transferred into the leaf tissue by agroinfiltration according to two processes. For the production of small batches of pectate lyases intended for prototype screening, the agrobacteria are injected manually by virtue of a syringe applied to the epidermis of the lower surface of the leaf. Leaf disks taken from the leaves 4 to 6 days after agroinfiltration are used for the analysis of the different prototypes of pectate lyases. This screening step makes it possible to define the expression vector which will be used to obtain a pectate lyase of optimal quality. The same process is used for large-scale commercial production, but in this case the agroinfiltration is carried out under vacuum, in chambers containing several liters of an agrobacterial culture and in which several tens of plants are infiltrated simultaneously. These plants are then placed back in culture for 4 to 6 days before the purification of the pectate lyases from the leaf extracts (FIG. 2).

Expression of the Pectate Lyase Amb a 1 and Its Subunits

The expression of the proteins and also the yields are analyzed by Western blotting and ELISA, respectively. The results are illustrated for the three forms of cDNA encoding the natural protein (FIG. 3).

The expressed proteins are active. Indeed, the expression of Amb a 1 causes large necroses from the 4th day of expression, compared to control plants. These necroses are due to high pectate lyase activity (Liu et al., 2010).

Purification and Characterization

As illustrated in FIG. 4, the pectate lyases are extracted from fresh or frozen biomass then purified on IMAC column (HisTrap Excell).

This purification enables the production of the precursor form (preproprotein) and of the alpha subunit.

Finally, as shown in FIG. 5, it is possible to combine the allergens Amb a 1 and Amb a 11 obtained by the process according to the invention, to obtain a composition which may be used in allergy immunotherapy. These allergens may be produced and purified after mechanical extraction as described in application FR1255510, or according to the alternative of extraction by enzymatic infiltration as described in the present application.

1-17. (canceled)

18. A plant cell comprising a DNA molecule comprising at least one heterologous nucleotide sequence encoding a preproprotein of a pectate lyase chosen from Amb a 1, the alpha subunit of Amb a 1, the beta subunit of Amb a 1, and homologs of Amb a 1, functionally bound to a strong promoter.

19. The plant cell as claimed in claim 18, wherein said DNA molecule is not integrated into the plant cell genome.

20. The plant cell as claimed in claim 18, wherein the pectate lyase is chosen from Amb a 1.0101, Amb a 1.0201, Amb a 1.0202, Amb a 1.0301, Amb a 1.0302, Amb a 1.0303, Amb a 1.0304, Amb a 1.0305, Amb a 1.0401, Amb a 1.0402, Amb a 1.0501, Amb a 1.0502, the alpha subunit of Amb a 1, the beta subunit of Amb a 1 and proteins of the Asteraceae family which have at least 57% identity, preferably at least 60% identity, preferably at least 64% identity, with one of the isoforms of Amb a 1.

21. The plant cell as claimed in claim 18, wherein the pectate lyase is chosen from Amb a 1.0101, Amb a 1.0201, Amb a 1.0202, Amb a 1.0301, Amb a 1.0302, Amb a 1.0303, Amb a 1.0304, Amb a 1.0305, Amb a 1.0401, Amb a 1.0402, Amb a 1.0501, Amb a 1.0502, the alpha subunit of Amb a 1, the beta subunit of Amb a 1 and proteins of Artemisia vulgaris, Ambrosia psilostachya or Ambrosia trifida which have at least 57% identity, preferably at least 60% identity, preferably at least 64% identity, with one of the isoforms of Amb a 1.

22. The plant cell as claimed in claim 18, wherein the heterologous nucleotide sequence comprises a sequence encoding the signal peptide, a sequence encoding the propeptide and a sequence encoding the mature pectate lyase.

23. The plant cell as claimed in claim 22, wherein:
the signal peptide is chosen from the natural pectate lyase signal peptide and the tobacco chitinase signal peptide; and
the propeptide is chosen from the natural pectate lyase propeptides and the propeptides of peptidases C1A.

24. The plant cell as claimed in claim 18, wherein the heterologous nucleotide sequence also comprises an intracellular trafficking peptide sequence which targets the pectate lyase in soluble or membrane form to the endoplasmic reticulum or the various compartments which constitute the endomembrane secretory system of the plant cell.

25. The plant cell as claimed in claim 18, which comprises an expression vector comprising:
elements of prokaryotic DNA encoding a bacterial origin of replication and a gene for antibiotic resistance;
the heterologous nucleotide sequence as previously defined;
an expression cassette allowing the expression of a silencing suppressor, preferably p19; and
DNA elements which control transcript processing, such as termination/polyadenylation sequences, preferably the Tnos sequence.

26. A plant comprising at least one plant cell as claimed in claim 18.

27. A process for producing a pectate lyase chosen from Amb a 1, the alpha subunit of Amb a 1, the beta subunit of Amb a 1, and homologs of Amb a 1, comprising the expression of said pectate lyase in a plant cell as claimed in claim 18 or in a plant comprising at least one said plant cell.

28. The process for producing a pectate lyase as claimed in claim 27, comprising the following steps:
a) transformation of agrobacteria with an expression vector comprising a heterologous nucleotide sequence encoding a preproprotein of a pectate lyase, functionally bound to a strong promoter; and
b) transfection of the plant cell or of the plant with the agrobacteria obtained in step a).

29. The process for producing a pectate lyase as claimed in claim 28, wherein the agrobacteria used in step a) are chosen from the strains LBA4404, GV3101, EHA 101/105 and C58, and wherein the transfection of step b) preferably comprises the following steps:
b1) culturing the plant cell or plant aeroponically or hydroponically, under LED lighting, preferably for five weeks hydroponically on free floats,
b2) agroinfiltration of the plant cell or plant obtained in b1), under vacuum, by the agrobacteria obtained in step a); and
b3) returning the plant cell or plant obtained in b2) to culturing, typically for 3 to 6 days.

30. The process for producing a pectate lyase as claimed in claim 28, also comprising a step c) of extraction of the pectate lyase produced, said step c) comprising the following steps:
infiltration under vacuum of the plant cell or leaves (i.e. the aerial part) of the plant, in an enzymatic solution comprising a pectinase or maceroenzyme, which does not have proteolytic activity,
taking off the infiltrated plant cell or infiltrated leaves from the plant and incubation, in an enzymatic solution of pectinase or maceroenzyme, for a duration of between 2 h 30 and 5 h, at a temperature of between 24° C. and 30° C., then
placing the mixture obtained under agitation at between 20 and 30 rpm at room temperature for a duration of between 30 minutes and 2 h,
filtration then optional centrifugation of the digestate obtained, and
recovery of the supernatant.


SEQUENCE LISTING (9 sequences)

SEQ ID NO: 1 - DNA, 1254 bp, Artificial Sequence
  cDNA encoding the natural preprotein (of sequence SEQ ID NO 7)
tctagaggtaccatggggatcaaacactgttgttacatcttgtattttaccttagcccttgtcactttgctgcaacctgttcgttctgccgaagatctccaggaaatcttaccagttaacgaaacaaggaggctgacaacaagtggagcatacaacattatagacgggtgctggaggggcaaagccgattgggcggaaaaccgaaaagcgttagccgattgtgcccaaggttttgggaagggaacagtgggcggaaaagatggtgatatatacacggtcaccagtgagctagatgatgatgttgcaaatccaaaagaaggcacactccggtttggtgccgcccaaaacaggcccttgtggatcatttttgaaagagatatggtgattcgtttggataaagagatggtggtaaacagtgacaagaccatcgatggccgaggggcgaaagttgaaatcattaacgctggtttcacccttaatggtgtcaagaatgtaatcattcataacataaatatgcatgatgttaaagtgaatccaggaggcctgattaagtccaacgatggtccagcagctccaagagctggtagtgatggtgatgctataagtatttctggtagttcacaaatatggatcgaccattgttcgctcagtaagtctgttgatgggctggtagatgccaagctcggcaccacacgcttaaccgtttccaacagcttattcacccaacaccagtttgtactattattcggggctggtgacgaaaatattgaagatagaggcatgctagcaacggtcgctttcaacacgttcactgataacgttgaccaaagaatgcctagatgtcggcatgggtttttccaagtcgttaacaacaactatgataaatggggatcgtatgccatcggtggtagcgcgtccccaaccatactcagccaagggaacagattctgcgcccccgatgaacgcagcaagaaaaatgtcctaggaaggcatggtgaagccgccgcagagtcgatgaagtggaactggagaacgaataaagacgtgcttgaaaatggtgctatttttgttgcatccggggtcgatccagtgctaacccctgagcaaagcgcagggatgattccagccgaaccaggagagtccgctctaagcctcactagtagtgctggtgtactctcatgccaacccggagcaccttgccatcatcatcatcatcatgattataaagatgatgatgataaagtttgagtcgac

SEQ ID NO: 2 - DNA, 1254 bp, Artificial Sequence
  cDNA optimized for a use in N.benthamiana, encoding the natural preprotein (of sequence SEQ ID NO 7)
tctagaggtaccatgggcatcaagcactgctgctacatcctctacttcaccctcgccctcgtcaccctcctccagcccgtccgctccgccgaggacctccaggagatcctccccgtcaacgagacccgccgcctcaccacctccggcgcctacaacatcatcgacggctgctggcgcggcaaggccgactgggccgagaaccgcaaggccctcgccgactgcgcccagggcttcggcaagggcaccgtcggcggcaaggacggcgacatctacaccgtcacctccgagctcgacgacgacgtcgccaaccccaaggagggcaccctccgcttcggcgccgcccagaaccgccccctctggatcatcttcgagcgcgacatggtcatccgcctcgacaaggagatggtcgtcaactccgacaagaccatcgacggccgcggcgccaaggtcgagatcatcaacgccggcttcaccctcaacggcgtcaagaacgtcatcatccacaacatcaacatgcacgacgtcaaggtcaaccccggcggcctcatcaagtccaacgacggccccgccgccccccgcgccggctccgacggcgacgccatctccatctccggctcctcccagatctggatcgaccactgctccctctccaagtccgtcgatggcctcgtcgatgccaagctcggcaccacccgcctcaccgtctccaactccctcttcacccagcaccagttcgtcctcctcttcggcgccggcgacgagaacatcgaggaccgcggcatgctcgccaccgtcgccttcaacaccttcaccgacaacgttgaccagcgcatgccccgctgccgccacggcttcttccaggtcgtcaacaacaactacgacaagtggggctcctacgccatcggcggctccgcctcccccaccatcctctcccagggcaaccgcttctgcgcccccgacgagcgctccaagaagaacgtcctcggccgccacggcgaggccgccgccgagtccatgaagtggaactggcgcaccaacaaggacgtcctcgagaacggcgccatcttcgtcgcctccggcgttgaccccgtcctcacccccgagcagtccgccggcatgatccccgccgagcccggcgagtccgccctctccctcacctcctccgccggcgtcctctcctgccagcccggcgccccctgccatcatcatcatcatcatgattataaagatgatgatgataaagtttgagtcgac

SEQ ID NO: 3 - DNA, 1254 bp, Artificial Sequence
  harmonized cDNA encoding the natural form of the preprotein (of sequence SEQ ID NO 7)
tctagaggtaccatggggattaaacactgttgttacattttgtactttactctcgcactcgtgaccctcctccagcctgtgcgctccgcagaggatcttcaggagattctcccagttaatgagaccaggaggctgaccacctccggagcatacaatattatcgacggatgctggaggggaaaggcagattgggccgagaatcggaaggccctcgcagattgcgcacaaggttttggcaagggaaccgtgggaggaaaggatggtgatatctacaccgtgacttccgagctcgatgatgatgttgcaaatccaaaggagggaaccctccgcttcggtgcagcacagaataggcccctctggattatttttgagagggatatggtgattcggctcgataaggagatggtggtcaattccgacaagactattgatggacggggagccaaggttgagattattaatgctggtttcactctcaatggtgttaagaatgtcattattcataatatcaatatgcacgatgttaaggtgaatcctggaggactgattaagtccaatgatggtcctgcagctcctagagctggttccgatggtgatgctatctccatttccggttcctcccagatctggattgaccattgttccctttccaagtccgtggatggtctcgtcgatgctaagcttggaactacccgcctcactgtttccaattccctcttcactcagcaccagtttgtcctcctcttcggcgctggtgacgagaatattgaggataggggtatgctcgcaaccgtggctttcaataccttcaccgataatgttgaccagaggatgcctaggtgccggcatggatttttccaggtggttaataataattacgataagtggggatcctacgcaattggtggttccgcctcccctactatcctttcccagggaaataggttctgcgcacccgatgagcgctccaagaagaatgtgctcggaaggcacggtgaggcagcagcagagtccatgaagtggaattggaggaccaataaggacgtgctcgagaatggtgctatttttgttgcatccggagttgatcctgtgctcactcctgagcagtccgcaggaatgattcctgcagagcctggagagtccgctctctcccttacctcctccgctggtgtcctttcctgccagcccggagcaccttgccatcatcatcatcatcatgattataaagatgatgatgataaagtttgagtcgac

SEQ ID NO: 4 - DNA, 606 bp, Artificial Sequence
  Native/optimized/harmonized cDNA encoding the beta subunit
tctagaggtaccatggggatcaaacactgttgttacatcttgtattttaccttagcccttgtcactttgctgcaacctgttcgttctgccgaagatctccaggaaatcttaccagttaacgaaacaaggaggctgacaacaagtggagcatacaacattatagacgggtgctggaggggcaaagccgattgggcggaaaaccgaaaagcgttagccgattgtgcccaaggttttgggaagggaacagtgggcggaaaagatggtgatatatacacggtcaccagtgagctagatgatgatgttgcaaatccaaaagaaggcacactccggtttggtgccgcccaaaacaggcccttgtggatcatttttgaaagagatatggtgattcgtttggataaagagatggtggtaaacagtgacaagaccatcgatggccgaggggcgaaagttgaaatcattaacgctggtttcacccttaatggtgtcaagaatgtaatcattcataacataaatatgcatgatgttaaagtgaatccaggaggcctgattaagcatcatcatcatcatcatgattataaagatgatgatgataaagtttgagtcgac

SEQ ID NO: 5 - DNA, 786 bp, Artificial Sequence
  Native/optimized/harmonized cDNA encoding the alpha subunit
tctagaggtaccatggggatcaaacactgttgttacatcttgtattttaccttagcccttgtcactttgctgcaacctgttcgttctaacgatggtccagcagctccaagagctggtagtgatggtgatgctataagtatttctggtagttcacaaatatggatcgaccattgttcgctcagtaagtctgttgatgggctggtagatgccaagctcggcaccacacgcttaaccgtttccaacagcttattcacccaacaccagtttgtactattattcggggctggtgacgaaaatattgaagatagaggcatgctagcaacggtcgctttcaacacgttcactgataacgttgaccaaagaatgcctagatgtcggcatgggtttttccaagtcgttaacaacaactatgataaatggggatcgtatgccatcggtggtagcgcgtccccaaccatactcagccaagggaacagattctgcgcccccgatgaacgcagcaagaaaaatgtcctaggaaggcatggtgaagccgccgcagagtcgatgaagtggaactggagaacgaataaagacgtgcttgaaaatggtgctatttttgttgcatccggggtcgatccagtgctaacccctgagcaaagcgcagggatgattccagccgaaccaggagagtccgctctaagcctcactagtagtgctggtgtactctcatgccaacccggagcaccttgccatcatcatcatcatcatgattataaagatgatgatgataaagtttgagtcgac

SEQ ID NO: 6 - protein, 80 aa, Artificial Sequence
  propeptide Der p1
RPSSIKTFEEYKKAFNKSYATFEDEEAARKNFLESVKYVQSNGGAINHLSDLSLDEFKNRFLMSAEAFEHLKTQFDLNAE

SEQ ID NO: 7 - protein, 417 aa, Ambrosia artemisiifolia
SRGTMGIKHCCYILYFTLALVTLLQPVRSAEDLQEILPVNETRRLTTSGAYNIIDGCWRGKADWAENRKALADCAQGFGKGTVGGKDGDIYTVTSELDDDVANPKEGTLRFGAAQNRPLWIIFERDMVIRLDKEMVVNSDKTIDGRGAKVEIINAGFTLNGVKNVIIHNINMHDVKVNPGGLIKSNDGPAAPRAGSDGDAISISGSSQIWIDHCSLSKSVDGLVDAKLGTTRLTVSNSLFTQHQFVLLFGAGDENIEDRGMLATVAFNTFTDNVDQRMPRCRHGFFQVVNNNYDKWGSYAIGGSASPTILSQGNRFCAPDERSKKNVLGRHGEAAAESMKWNWRTNKDVLENGAIFVASGVDPVLTPEQSAGMIPAEPGESALSLTSSAGVLSCQPGAPCHHHHHHDYKDDDDKVVD

SEQ ID NO: 8 - protein, 201 aa, Ambrosia artemisiifolia
SRGTMGIKHCCYILYFTLALVTLLQPVRSAEDLQEILPVNETRRLTTSGAYNIIDGCWRGKADWAENRKALADCAQGFGKGTVGGKDGDIYTVTSELDDDVANPKEGTLRFGAAQNRPLWIIFERDMVIRLDKEMVVNSDKTIDGRGAKVEIINAGFTLNGVKNVIIHNINMHDVKVNPGGLIKHHHHHHDYKDDDDKVVD

SEQ ID NO: 9 - protein, 261 aa, Ambrosia artemisiifolia
SRGTMGIKHCCYILYFTLALVTLLQPVRSNDGPAAPRAGSDGDAISISGSSQIWIDHCSLSKSVDGLVDAKLGTTRLTVSNSLFTQHQFVLLFGAGDENIEDRGMLATVAFNTFTDNVDQRMPRCRHGFFQVVNNNYDKWGSYAIGGSASPTILSQGNRFCAPDERSKKNVLGRHGEAAAESMKWNWRTNKDVLENGAIFVASGVDPVLTPEQSAGMIPAEPGESALSLTSSAGVLSCQPGAPCHHHHHHDYKDDDDKVVD